(19) United States
(12) Patent Application Publication
(10) Pub. No.: US 2026/0167868 A1
(43) Pub. Date: Jun. 18, 2026

(54) SYSTEMS AND METHODS FOR PROCESSING BIOMASS

(71) Applicant: CHAR Technologies Research Inc., Toronto (CA)

(72) Inventors: Andrew J. White, Toronto (CA); Kenneth P. Goodboy, Franklin Park, PA (US); Oussama M. Hamdanie, Toronto (CA); Stefano Tacchino, Milton (CA)

(21) Appl. No.: 19/533,833

(22) Filed: Feb. 9, 2026

Related U.S. Application Data
(63) Continuation of application No. PCT/CA2024/051042, filed on Aug. 8, 2024.
(60) Provisional application No. 63/531,601, filed on Aug. 9, 2023.

(51) Int. Cl. C10B 53/02; C10B 1/10

(52) U.S. Cl. CPC C10B 53/02; C10B 1/10; C10G 2300/1011; C10G 2300/1014

(57) ABSTRACT

A method of processing biomass is disclosed. The method includes providing biomass to a pyrolysis kiln, where the biomass has a moisture content of less than 40% and an ash content of less than 50%, and where the biomass has a biomass input temperature of less than 125° C. The method continues with heating at least an upstream portion of the pyrolysis kiln to maintain an upstream kiln temperature of between 800° C. and 1250° C., and feeding the biomass through the pyrolysis kiln for a pyrolysis duration of 5 to 60 minutes to produce a kiln output that includes biocarbon and syngas. The biomass increases from the biomass input temperature to a biomass reaction temperature of at least 800° C. in less than 25% of the pyrolysis duration. The method continues with discharging at least the biocarbon and the syngas from the pyrolysis kiln. Other methods and pyrolysis kilns are also disclosed.

### FIELD

The field of the invention relates to systems and methods for processing biomass. Specifically, this application relates to systems and methods that include a pyrolysis kiln for converting biomass into biocarbon and syngas.

INTRODUCTION

Pyrolysis is the heating of organic material, such as biomass, in the absence of oxygen. Pyrolysis, within a pyrolysis kiln, may convert biomass into biocarbon (e.g., biochar and/or biocoal), syngas, and bio-oil. Biocarbon is a carbon-rich and porous material which can be used for a wide range of applications, such as, for example, as a soil additive, as a filtration media, as an animal feed additive, as biocoal for metallurgical coal replacement in heavy industrial processes, etc. Syngas is a primarily a mixture of hydrogen, methane, carbon monoxide, carbon dioxide, water vapor, and light hydrocarbons (C₂ and C₃) and can be used as a fuel gas directly, but also as a base for generating RNG (Renewable Natural Gas). Bio-oil may also be used as a fuel and is a complex mixture of organic liquids that can contain as many as 70 or more hydrocarbons.

### SUMMARY

In one aspect, a method of processing biomass is provided. The method comprises:
providing biomass to a pyrolysis kiln, the biomass having a moisture content of less than 40% and ash content of less than 50%, the biomass having a biomass input temperature of less than 125° C.;
heating at least an upstream portion of the pyrolysis kiln to maintain an upstream kiln temperature of between 800° C. and 1250° C., and feeding the biomass through the pyrolysis kiln for a pyrolysis duration of 5 to 60 minutes to produce an kiln output comprising biocarbon and syngas, the biomass increasing from the biomass input temperature to a biomass reaction temperature of at least 800° C. in less than 25% of the pyrolysis duration; and
discharging at least the biocarbon and the syngas from the pyrolysis kiln.

In another aspect, another example of a method of processing biomass is provided which comprises:
providing biomass to a pyrolysis kiln, the biomass having a moisture content of less than 40% and ash content of less than 50%;
heating at least an upstream portion of the pyrolysis kiln containing the biomass to maintain an upstream kiln temperature of between 800° C. and 1250° C., and feeding the biomass through the pyrolysis kiln for a pyrolysis duration of 5 to 60 minutes to produce a kiln output comprising at least biocarbon and syngas,
   the kiln output, excluding any ash, having an output composition comprising greater than 19% by weight biocarbon, greater than 50% by weight syngas, and less than 20% by weight bio-oil,
discharging at least the biocarbon and the syngas from the pyrolysis kiln.

In another aspect, another example of a method of processing biomass is provided which comprises:
providing biomass to a pyrolysis kiln, the biomass having a moisture content of less than 40% by weight and an ash content less than 50%;
heating at least an upstream portion of the pyrolysis kiln containing the biomass to maintain an upstream kiln temperature of between 800° C. and 1250° C., and feeding the biomass through the pyrolysis kiln for a pyrolysis duration of 5 to 60 minutes to produce a kiln output comprising biocarbon and syngas; and
feeding the syngas to an olivine reactor as reactor input syngas, the reactor input syngas having an input H₂ concentration; and
heating the olivine reactor containing the syngas to maintain a reactor temperature of between 850° C. and 1250° C. to produce reactor output syngas, the reactor output syngas having an output H₂ concentration that is at least 200% of the input H₂ concentration.

In yet another aspect, an example of a pyrolysis kiln is provided. The pyrolysis kiln comprises:
a heat tube having an outer heat tube surface, the heat tube extending longitudinally from an upstream heat tube end to a downstream heat tube end along a heat tube rotation axis, the heat tube having a heat tube inlet at the upstream heat tube end and a heat tube outlet at the downstream heat tube end, the heat tube rotation axis and gravity direction defining a heat tube bisecting plane;
at least three first burners longitudinally spaced apart on a first side of the heat tube bisecting plane, the first burners positioned and oriented to apply heat to the heat tube below the heat tube rotation axis; and
at least three second burners longitudinally spaced apart on a second side of the heat tube bisecting plane, the second burners positioned and oriented to apply heat to the heat tube below the heat tube rotation axis.

### DRAWINGS

For a better understanding of the described embodiments and to show more clearly how they may be carried into effect, reference will now be made, by way of example, to the accompanying drawings in which:

FIG. 1 is a flowchart illustrating a method for processing biomass in accordance with an embodiment;

FIG. 2 is a schematic illustrating a system for processing biomass in accordance with an embodiment;

FIG. 3 is a perspective view of an embodiment of a pyrolysis kiln; and

FIG. 4 is a cross-sectional view of the pyrolysis kiln of FIG. 3, taken along line 4-4.

The drawings included herewith are for illustrating various examples of articles, methods, and apparatuses of the teaching of the present specification and are not intended to limit the scope of what is taught in any way.

### DESCRIPTION OF VARIOUS EMBODIMENTS

Numerous embodiments are described in this application and are presented for illustrative purposes only. The described embodiments are not intended to be limiting in any sense. The invention is widely applicable to numerous embodiments, as is readily apparent from the disclosure herein. Those skilled in the art will recognize that the present invention may be practiced with modification and alteration without departing from the teachings disclosed herein. Although particular features of the present invention may be described with reference to one or more particular embodiments or figures, it should be understood that such features are not limited to usage in the one or more particular embodiments or figures with reference to which they are described.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising” and variations thereof mean “including but not limited to,” unless expressly specified otherwise. A listing of items does not imply that any or all of the items are mutually exclusive, unless expressly specified otherwise. The terms “a,” “an” and “the” mean “one or more,” unless expressly specified otherwise.

As used herein and in the claims, a group of elements are said to “collectively” perform an act where that act is performed by any one of the elements in the group, or performed cooperatively by two or more (or all) elements in the group.

As used herein and in the claims, a first line or axis is said to be “perpendicular” to a second line or axis in three-dimensional space when the second line or axis is parallel to or collinear with an imaginary line that intersects the first line at a 90-degree angle, or within an angle of about 5 degrees of parallel to or collinear with the imaginary line.

As used herein and in the claims, a first element is said to extend “transverse” to a second element, where the first element extends within 45 degrees of perpendicular to the second element.

Some elements herein may be identified by a part number, which is composed of a base number followed by an alphabetical or subscript-numerical suffix (e.g., 112a, or 112₁). Multiple elements herein may be identified by part numbers that share a base number in common and that differ by their suffixes (e.g., 112₁, 112₂, and 112₃). All elements with a common base number may be referred to collectively or generically using the base number without a suffix (e.g., 112).

It should be noted that terms of degree such as “substantially”, “about”, and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

### General Description of a Method for Processing Biomass

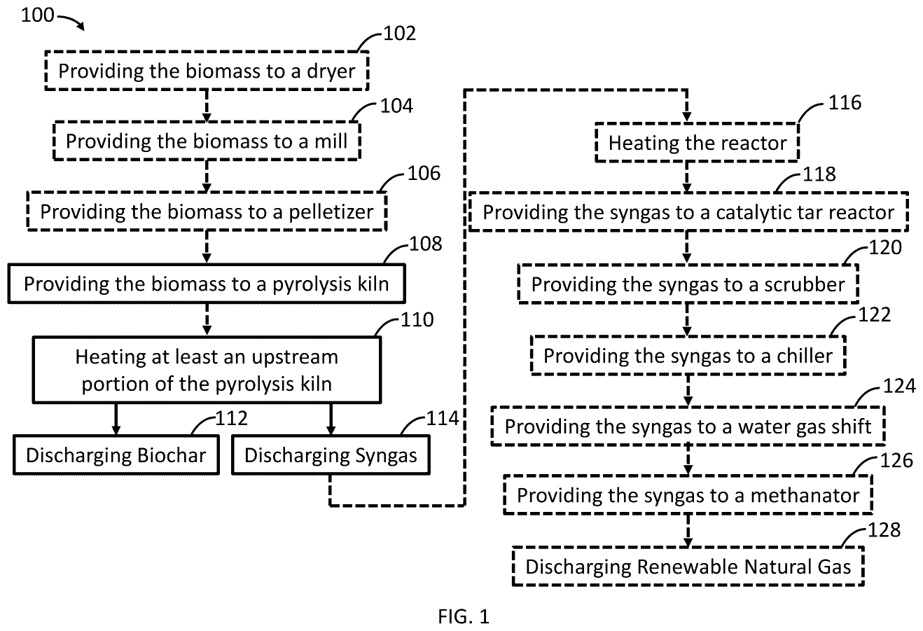

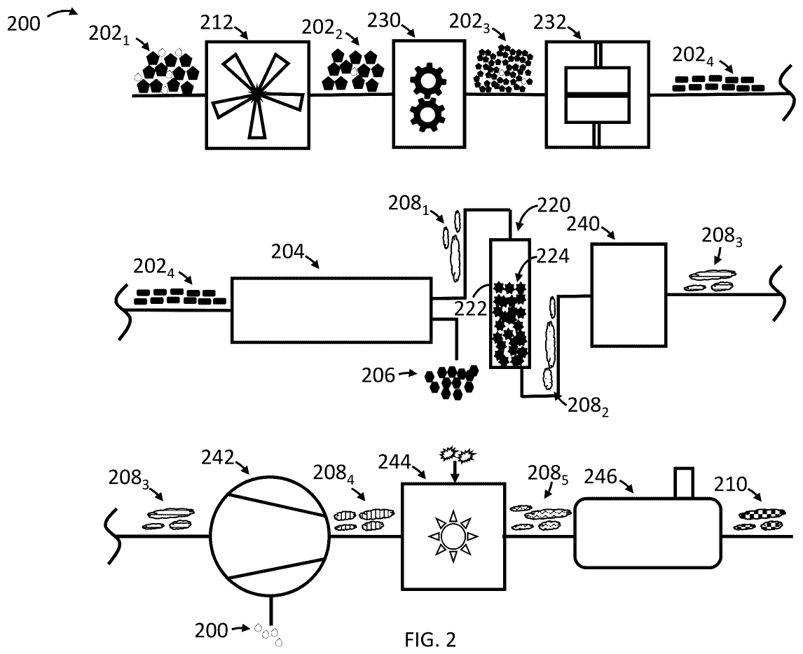

FIG. 1 shows a flow chart illustrating a method 100 for processing biomass. It is to be understood that the method 100 shown in FIG. 1 is exemplary, and some of the steps shown therein may be optional, as described in more detail below. FIG. 2, schematically illustrates an example of a system 200 for processing biomass 202 (i.e., a system able to carry out the method 100).

Pyrolysis of biomass may produce biocarbon, syngas, and bio-oil. Factors such as the type of biomass, heating rate, kiln temperature, pyrolysis duration, biomass particle size, bed depth, water vapor concentration all contribute to the weight percentage and quality of each of biocarbon, syngas, and bio-oil produced when processing biomass in a pyrolysis kiln. In the description that follows, system and methods that maximize biocarbon and syngas production while minimizing bio-oil production are described. Specifically, systems and methods for producing biocarbon having high fixed carbon content and syngas having high hydrogen, methane, and carbon monoxide; and low carbon dioxide, and light hydrocarbons (C₂-C₃) are described.

Referring to FIGS. 1 and 2, method 100 for processing biomass may start at step 108 in which biomass 202 is provided to a pyrolysis kiln 204. In the example illustrated in FIG. 2, the biomass 202 is provided to the pyrolysis kiln 204 as pelletized biomass 202₄. In other examples, the biomass 202 may not be pelletized prior to being provided to the pyrolysis kiln 204.

The pyrolysis kiln 204 is an indirect-fired kiln suitable for transforming biomass 202 into at least one of biocarbon 206, syngas 208, and bio-oil (not shown). Indirect-fired means that the heat is supplied on the outside of the heat tube 256. A particular example of a pyrolysis kiln 204 that may be used in method 100 is described in more detail below.

The biomass 202 provided to the pyrolysis kiln 204 at step 108 may have a moisture content of less than 20% by weight. In some examples, the biomass 202 provided to the pyrolysis kiln 204 may have a moisture content of less than 40% by weight. It may be desirable to provide the biomass 202 with a moisture content less than 20% because a higher moisture content may require greater heat input to convert the biomass 202 into at least one of biocarbon 206, syngas 208, and bio-oil, which may reduce the economic efficiency of the process. But a certain amount of moisture (e.g. at least 10%, such as 10 to 40%) in the biomass is desirable to convert carbon monoxide into more valuable hydrogen via the water gas shift reaction which is CO+H₂O=H₂+CO₂. Any type of biomass comprising of cellulose, hemi-cellulose, lignin, and other organic compounds in varying ratios will suffice for this process but biomasses with low ash content are preferred, as ash cannot be converted into syngas for example. Woody biomass is preferred due to its higher density, but agricultural wastes and residuals such as sugar bagasse, rice husks, corn cobs, nut shells (almond, pistachio, walnut), anaerobic or acidogenic digestate (produced from manures, food wastes, agricultural wastes, and residues in any combination), and biosolids from municipal waste for example can be used. Preferably, the ash content of biomass 202 is less than 50% to increase the potential quantity of useful output from method 100.

As shown in FIG. 1, to reduce the moisture content of the biomass 202 provided to the pyrolysis kiln 204, at optional step 102, the biomass 202 may be provided as moist biomass 202₁ to a dryer 212 upstream of the pyrolysis kiln 204. Within the dryer 212, the biomass 202 may be dried from an initial moisture content to a moisture content less than, for example, 15% by weight. Accordingly, dried biomass 202₂ may be produced by the dryer 212. It may not be necessary to provide the biomass 202 to an upstream dryer 212 when the moisture content of the biomass 202 provided to the processing facility already has a desirable moisture content (e.g., less than 20% or less than 15% by weight).

Referring back to step 108, the biomass 202 provided to the pyrolysis kiln 204 may have an ambient input temperature which is normally less than 40° C. However, a warmer input temperature is generally beneficial as it will require less energy consumption to heat the biomass 202 to the target temperature within pyrolysis kiln 204. Woody biomass typically holds moisture within the cellulose, hemicellulose, and lignin of the wood. Digestate may include free water (i.e., water that is not part of the biomass structure). It may be economically desirable that the ambient temperature is not less than freezing, as free water will form ice and require additional energy to melt. The input temperature of the biomass 202 provided to the pyrolysis kiln 204 may be impacted by optional upstream processing steps (e.g., drying) and/or operating temperatures within the processing facility. In some example, drying can result in an input temperature of over 75° (e.g. 80-115° C.) and a pellet mill may produce pellets that are over 90° C. (e.g. 100-115° C.) in temperature due to the input of mechanical energy in the pellet manufacturing process. Accordingly, in some embodiments, biomass 202 provided to pyrolysis kiln 204 may have an input temperature of less than 125° C. (e.g. 5° C. to 125° C.).

The pyrolysis kiln 204 receiving the biomass 202 in step 108 is maintained at a kiln temperature suitable to convert the biomass 202 into at least one of biocarbon 206, syngas 208, and bio-oil as it passes through the pyrolysis kiln 204. As described in more detail below, the kiln temperature may vary along the length of the pyrolysis kiln 204. It has been determined that the composition of the pyrolysis kiln output material (i.e., percent by weight biocarbon 206, percent by weight syngas 208, and percent by weight bio-oil) is a function of the temperature of the pyrolysis kiln 204 and retention time of the biomass 202 within the pyrolysis kiln 204. Further, it has been determined that the composition of the pyrolysis kiln output material is affected by the rate at which the input feed of biomass 202 is heated. Further yet, it has been determined that the composition of the pyrolysis kiln output material is affected by the rate at which the biomass 202 (biocarbon) is cooled as it passes through a downstream portion of the pyrolysis kiln 204 and/or after it exits the pyrolysis kiln 204.

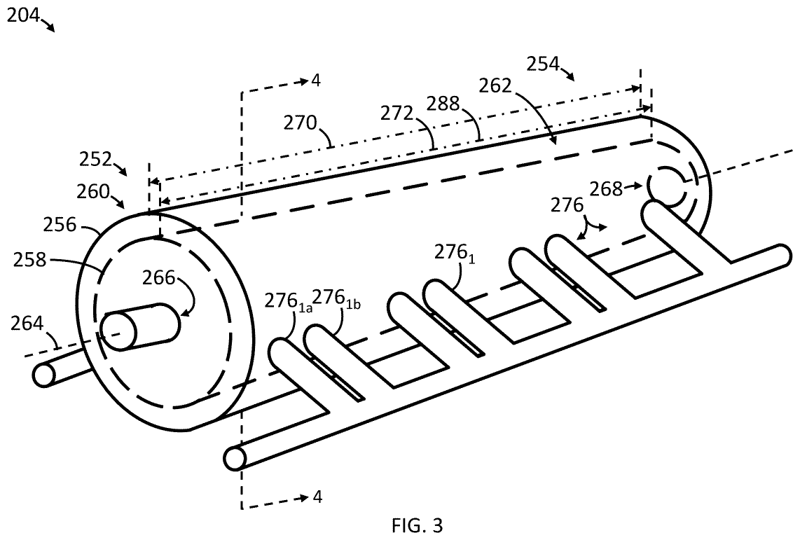

At step 108 of method 100, at least an upstream portion 252 (see, e.g., FIG. 3) of the pyrolysis kiln 204 containing the biomass 202 is heated to maintain an upstream kiln temperature of between 800° C. and 1250° C. In some examples, the pyrolysis kiln 204 containing the biomass 202 may be heated to maintain an upstream kiln temperature of between 900900° C. and 1000° C. For clarity, gasifiers may have temperatures in a similar range but their retention time is only a matter of seconds, and they produce primarily gas and little or no biocarbon. In contrast, indirect fired kilns operate in terms of minutes. In some examples, method 100 was performed with a primary syngas goal of forming hydrogen. In doing so, it was discovered that an upstream kiln temperature in the range of 800 to 1250° C. (and preferably 900 to 1000° C.) provided a volume or mole % of H₂ in the syngas that increased exponentially along with carbon monoxide, and provided a volume or mole % of undesirable carbon dioxide (having no fuel value) which decreased. This provided a more valuable syngas output.

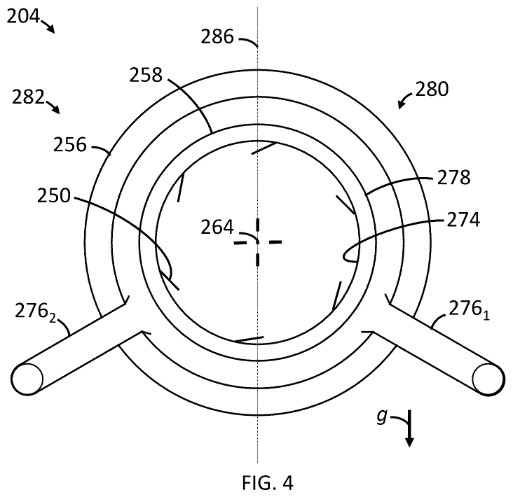

As used herein and in the claims, a kiln temperature (upstream and/or downstream) may be measured at an outer heat tube surface 278 of a heat tube 258 of the pyrolysis kiln 204 (see, e.g., FIG. 4). Based on the thermal properties of the heat tube 256, the biomass 202 within the pyrolysis kiln 204 (specifically within the heat tube 256 of the pyrolysis kiln 204) may be exposed to a temperature less than the kiln temperature. Kiln 204 has an upstream portion 252 and a downstream portion 254. Upstream portion 252 includes not more than the upstream half of kiln 204 (e.g., 10-50% of kiln 204), and downstream portion 254 includes not more than the downstream half of kiln 204 (e.g., 10-50% of kiln 204).

In some examples, the biomass 202 may be heated within the pyrolysis kiln 204 for a pyrolysis duration of 5 to 60 minutes. As used herein and in the claims, the pyrolysis duration is the time it takes for material to travel from a heat tube inlet 266 of the pyrolysis kiln 204 to a heat tube outlet 268 of the pyrolysis kiln 204. Optionally, the pyrolysis duration may be between 5 and 30 minutes. The pyrolysis duration may be adjusted by changing the rotational speed of the pyrolysis kiln 204. That is, in some examples, the faster the pyrolysis kiln 204 rotates, the shorter the pyrolysis duration will be. In some examples, as shown in FIG. 4, the pyrolysis kiln 204 may include flights 250 to urge the biomass 202 through the pyrolysis kiln 204. Alternatively or in addition to flights 250, the pyrolysis duration may be adjusted by an inclination of kiln 204, in which inlet 266 may be at a higher elevation than outlet 268 so that the biomass 202 is urged toward outlet 268 by gravity. If the kiln duration is too short, biomass will not be totally converted to syngas and biocarbon (i.e., low efficiency conversion of biomass to useful outputs), and/or the output biocarbon may have a volatile matter content that is too high (e.g., greater than 10%) for end use applications, such as metallurgical coal replacement. If the kiln duration time is too long, the process becomes less economically viable and the amount of output biocarbon may decrease from a reaction with excess water vapor.

It has been determined that heating the biomass 202 in a pyrolysis kiln 204 having an upstream kiln temperature between 800° C. and 1250° C. for a pyrolysis of duration of 5 to 60 minutes (more preferably 5 to 30 minutes) will allow the biomass 202 to transform into at least one of biocarbon 206, syngas 208, and bio-oil. Specifically, it has been determined that operating the pyrolysis kiln 204 under these conditions maximizes biocarbon 206 and syngas 208 production while minimizing bio-oil production.

In some examples, the biomass 202 may be heated within the pyrolysis kiln 204 so that the biomass 202 increases from the biomass input temperature to a biomass reaction temperature of at least 800° C. in less than 25% of the pyrolysis duration. The biomass reaction temperature is the temperature at which the biomass begins transforming into at least one of biocarbon 206, syngas 208, and bio-oil. Without being limited by theory, it is believed that such rapid heating quickly releases the moisture content (water) and VM (organic Volatile Matter) of the biomass so that it has more time to continue reacting in the vapor phase for syngas production of the desired hydrogen, methane, and carbon monoxide while decreasing the undesirable carbon dioxide, C₂-C_{3,} and bio-oils and tar.

Heating the biomass 202 in a pyrolysis kiln 204 having an upstream kiln temperature between 800° C. and 1250° C. for a pyrolysis of duration of 5 to 60 minutes (e.g. 5 to 30 minutes) such that the biomass 202 increases from the biomass input temperature to a biomass reaction temperature of at least 800° C. in less than 25% of the pyrolysis duration may increase the percent by weight biocarbon 206 and percent by weight syngas 208 produced based on a fixed volume of biomass input. Without being limited by theory, it is believed that an increase in the amount of biocarbon is obtained from tars and other heavy hydrocarbons that crack, along with additional gas phase reactions, with the biocarbon and even being possibly a catalyst for said cracking. That is, for example, in known systems, an input of biomass 202 may turn into (apart from any ash) less than 18 (e.g. 8-18)% by weight biocarbon 206, less than 45% (e.g. 16-45%, average 34) by weight syngas 208, and typically 48% by weight bio-oil. However, when operating the pyrolysis kiln 204 according to the parameters outlined above, it has been found that the same volume of biomass 202 may turn into (apart from any ash) greater than 19% by weight biocarbon 206, greater than 50% (e.g. 50-60%) by weight syngas 208, and less than 20% by weight bio-oil.

In addition to increasing the percent by weight biocarbon 206 and percent by weight syngas 208 produced when operating the pyrolysis kiln 204 as described above, it has been determined that the syngas 208 produced and discharged from the pyrolysis kiln 204 may have a more desirable composition. For example, it has been found that the syngas 208 discharged from the pyrolysis kiln 204 may increase by 1.5 to 4.1X by volume hydrogen (e.g. to 18-68 vol% H₂), while carbon dioxide (CO₂) decreases from 18% to 3.5 vol % or lower in regards to the reference point of 625° C. (i.e. compared to performing the same process at a kiln temperature of 625° C.). Also, all C₂ and C₃ hydrocarbons have been found to be at or near zero volume % (e.g. 0.01 to 2% by volume). By comparison, known systems discharge syngas from a pyrolysis kiln having less than 43% by volume H₂ and greater than 19% by volume CO₂. Notably, the CO₂ concentration is much lower when operating pyrolysis kiln 204 as described here. CO₂ is a waste product. CO₂ has no energy value, and as such a lower concentration of CO₂ provides the syngas 208 with greater energy value. Additionally, reducing the CO₂ in the syngas will allow for a higher methane yield when the syngas is processed via catalytic methanation. The catalytic process requires 4 moles of H₂ when reacting with CO₂, but only 3 moles of H₂ when reacting with CO. As such, syngas with lower CO₂ content requires less H₂ for the catalytic process, and H₂ is commonly a limiting factor when there is high CO₂ content. Also, the syngas yield may increase from approximately 50% to approximately 75% while the biocarbon yield does not decrease or decrease significantly. Normally more severe operating conditions of the kiln i.e. higher temperature and/or longer residence time decreases the biocarbon yield.

As noted above, in some examples, the upstream kiln temperature may be maintained between 900° C. and 1000° C. and the pyrolysis duration may be between 5 and 30 minutes. The specific combination of kiln temperature and pyrolysis duration that will help maximize biocarbon and syngas output may depend on the composition of biomass 202 provided to the pyrolysis kiln 204 and the targeted kiln output. For example, a lower downstream kiln temperature (e.g. to 625-850° C.) may be selected when the goal is to produce specific qualities and quantities of biocarbon. In another example, if the goal is to maximize syngas, then a kiln temperature of 900-1000° C. may be maintained throughout a length of the kiln. If the goal is to produce both syngas and biocarbon simultaneously, then a compromise on temperature and duration can be selected.

In some examples, the heat applied to the biomass 202 within the pyrolysis kiln 204 may vary along the length of the pyrolysis kiln 204. That is, in some embodiments, an upstream kiln temperature may be different than a downstream kiln temperature. For example, the upstream kiln temperature may be between 800° C. and 1250° C. while the downstream kiln temperature may be between 625° C. and 1000° C.

It has been determined that the downstream kiln temperature can affect the properties of the biocarbon 206 which is outputted from the pyrolysis kiln 204. For example, it has been determined that operating the pyrolysis kiln 204 with a downstream kiln temperature in the range of 625° C. and 850° C. may produce biocarbon 206 having a higher yield. As a second example, it has been determined that operating the pyrolysis kiln 204 with a downstream kiln temperature in the range of 800° C. and 1000° C., optionally between 800° C. and 900° C., may produce biocarbon 206 having a higher fixed carbon content.

Referring back to FIG. 1, at steps 112 and 114 biocarbon 206 and syngas 208 may be discharged from the pyrolysis kiln 204, respectively. The biocarbon 206 discharged from the pyrolysis kiln 204 may be packaged and shipped for further use. Optionally, as shown in FIG. 1, the syngas 208 discharged from the pyrolysis kiln 204 may be further refined within the processing facility. In other examples, the discharged syngas 208 may not be further refined.

As shown in FIGS. 1 and 2, upstream of the pyrolysis kiln 204, the biomass 202 may be subjected at least one of drying in a dryer 212 at step 102, fragmenting in hammermill 230 at step 106, and pelletizing in a pellet mill 232 at step 106.

When the biomass 202 is provided to a hammermill 230 upstream of the pyrolysis kiln 204, the biomass 202 may be fragmented into biomass particles 202₃ which have an average biomass particle size between 1 mm and 50 mm, which can be fed to the kiln as is. In some cases, if a hammermill is used to generate feed for a pellet mill, the particles from a hammermill need to be <50% of the diameter of the pellet mill in general. As an example, if the pellet mill produces ¼″ (6.35 mm) pellets, the largest particle should not be any larger than ⅛″ (3.18 mm) for such hammermills. As used herein and in the claims, a biomass particle having a biomass particle size between 1 mm and 50 mm will pass through an aperture having a 50 mm diameter. Optionally, the mill may fragment the biomass into biomass particles 202₃ having an average biomass particle size between 1 mm and 25 mm, 1 mm and 13 mm or between 1 mm and 3 mm. In pyrolysis reactors, a smaller particles size generally increases the available surface area, and therefore may provide a faster reaction rate. With a faster reaction rate, there may be greater productivity and therefore improved economics.

When the biomass 202 is provided to a pelletizer upstream of the pyrolysis kiln 204 and optionally downstream of the mill 230, the biomass 202 may be pelletized into biomass pellets having an average biomass pellet size between 5 mm and 7 mm. As used herein and in the claims, a biomass pellet having a biomass pellet size between 5 mm and 7 mm will pass through an aperture having a 5 and 7 mm diameter. Woody biomass, when dried to approximately 15% moisture can have a loose bulk density of 0.2-0.3 g/cc, but when processed via a pellet mill the loose bulk density can increase by a factor of up to 3.93 times depending on the wood (tree) type and starting particle size to generate a loose bulk density of 0.58-1.01 g/cc. Since a kiln is a volumetric tube, the higher the loose bulk density the more mass that can be throughput and thus the more favorable the economics.

As shown in FIG. 1, the syngas 208 discharged from the pyrolysis kiln 204 may be provided to a catalytic reactor 220 as reactor input syngas 208₁. Catalytic reactor 220 may reduce or eliminate bio-oil and tar from the reactor input syngas 208₁. As shown in FIG. 2, the catalytic reactor 220 may include a reactor body 222 that contains a volume of iron-based catalyst 224 (e.g. olivine) and optionally nickel. When provided to the catalytic reactor 220, the syngas 208 may pass-over the catalyst 224 contained within the reactor body 222. In catalysis, the gas is adsorbed upon the catalyst surface and the increase adsorbed concentration speeds up the rate of reactions. Therefore, the syngas must make intimate contact with the catalyst. The syngas goes through the bed of catalyst 224 (e.g., olivine) but cannot go through the catalyst itself.

It may be desirable to pass the syngas 208 through the catalytic reactor 220 (e.g., an olivine reactor) as it has been determined that doing so may increase the H₂ concentration of reactor output syngas 208₂ relative to reactor input syngas 208₁. That is, for example, it has been found that reactor output syngas 208₂ may have an output H₂ concentration that is at least 200% of the input H₂ concentration. For example, using wood as the input biomass 202₄, pyrolysis at 625° C. produces approximately 10 vol % H₂ but thermally reacting the same syngas at 900° C. produces approximately 28 vol % H₂. Continuing the example, passing the same syngas through olivine that is calcined at 900° C. to 1400° C. (e.g. 970° C.) for at least 3 hours (e.g. 5 hours), the H₂ concentration increases to approximately 33% (e.g., 30-35%). If the same syngas and olivine is not only calcined but reduced by H₂ (referred to as being “H₂ reduced”) at 700° C. for 2 hours, then the H₂ concentration becomes approximately 40 vol % (e.g., 37-43%). Not only may passing the syngas 208 through the catalytic reactor 220 increase the H₂ concentration, but it may also reduce or eliminate C₂-C₃ hydrocarbons (ethane, ethylene, propane, and propylene) and greatly decrease CO₂ in the discharged syngas 208. For example, reactor output syngas 208₂ may have an output CO₂ vol % that is less than 25% of the input CO₂ vol %. For the same conditions as H₂ in the examples above, CO₂ was approximately 23 vol % (when H₂ was approximately 10 vol %), approximately 16 vol % (when H₂ was approximately 28 vol %), approximately 13 vol % (when H₂ was approximately 33 vol %), and approximately 3 vol % (when H₂ was approximately 40 vol %) respectfully. In some embodiments, reactor output syngas 208₂ may have an output C₂-C₃ vol % that is less than 20% of the input C₂-C₃ vol %. For example, a reactor input syngas 208₁ with an input C₂-C₃ vol % of approximately 7 vol % may provide a reactor output syngas 208₂ with an output C₂-C₃ vol % of less than 1%. Notably, H₂ has high energy value and when reacted with CO, produces RNG. The extra hydrocarbons besides CH₄ can be a problem in the syngas scrubber and the CO Methanation process as they can form carbon deposits and poison the catalyst. Therefore, reducing these extra hydrocarbons (e.g., C₂-C₃ and bio-oil) can be very beneficial.

In some examples, the catalytic reactor 220 may be heated at step 116 to maintain a reactor temperature of between 850° C. and 1250° C. prior to providing syngas 208₁ to catalytic reactor 220. This temperature range may be effective for reducing or eliminating C₂-C₃ and bio-oil hydrocarbons from the syngas 208₁. Lower temperatures may be less effective or ineffective at reducing or eliminating C₂-C₃ hydrocarbons, and higher temperatures may have less favorable economy.

In some examples, the olivine 224 may be calcined before the reactor input syngas 208₁ is provided to the catalytic reactor 220. Exposing the reactor input syngas 208₁ to calcined olivine 224 may increase the H₂ concentration of the reactor output syngas 208₂ relative to reactor output syngas 208₂ that was not exposed to calcined olivine 224. That is, for example, when exposing syngas to non-calcined olivine, the H₂ concentration of the reactor output syngas 208₂ may be at least 200% of reactor input syngas 208₁ whereas when exposing syngas to calcined olivine, the H₂ concentration of the reactor output syngas 208₂ may be at least 300% of reactor input syngas 208₁. The percentage increases in H₂ will depend on the starting H₂. For example, higher kiln temperatures may produce reactor input syngas 208₁ with a higher starting H₂ (e.g. 30%) in which case the H₂ reactor output syngas 208₂ may not reach 300% of the H₂ in reactor input syngas 208₁ (e.g. 90%) but some lesser increase. Still, exposing the reactor input syngas 208₁ to calcined olivine 224 will typically increase the H₂ concentration of the reactor output syngas 208₂ relative to reactor output syngas 208₂ had it not been exposed to calcined olivine 224.

In addition to the above, steam may be added to the catalytic reactor 220 (e.g., olivine reactor). This may help reduce or eliminate C₂-C₃, bio-oil, and/or tar from the processed syngas 208.

In some embodiments, catalytic reactor 220 may be substituted by a substantially isothermal fluid bed CO/CO₂ Methanator.

Optionally, alternatively, or in addition to passing the syngas 208 through a catalytic reactor 220, the pyrolysis kiln 204 may have olivine 224 therein. It has been found that providing olivine 224 within the pyrolysis kiln 204 may increase the H₂ concentration and may reduce or eliminate C₂ and/or CO₂ within the syngas 208 discharged from the pyrolysis kiln 204.

As shown in FIGS. 1 and 2, in some examples, after passing the syngas 208 through the catalytic reactor 220, the syngas 208 may be further refined by any one or more (or all of):
(i) providing the syngas 208 to a scrubber 240 at step 120;
(ii) providing the syngas 208 to a chiller 242 at step 122;
(iii) providing the syngas 208 to at least one water gas shift 244 at step 124; and
(iv) providing the syngas 208 to at least one methanator 246 at step 126.

In some cases, two or more of these refinement steps may be achieved by one piece of equipment. For example, steps 124 and 126 (water gas shift and CO/CO₂ methanation) may be performed in a single fluid bed semi-isothermal reactor.

It may be desirable to provide the syngas 2082 to the scrubber 240 at step 120 to remove at least a portion of any residual bio-oils, as well as other potential contaminants (e.g., hydrogen sulfide (H₂S), hydrogen chloride (HCl), C₄⁺) and carbon fines (C) that may be present within the syngas 2082. In the scrubber 240, the object is to remove all sulfur compounds if possible. The CO Methanation catalyst may be elemental Nickel, in which case it may react immediately with H₂S and form NiS which is no longer active.

It may be desirable to provide the syngas 2083 to a chiller 242 at step 122 to remove water (H₂O) from the syngas 2083. In some examples, the chiller 242 may reduce the concentration of H₂O within the syngas 208 to less than 1.5% by volume. With a pre-reforming reactor before a water gas shift reactor, all or most of the water vapor is not required to be removed, as steam helps prevent carbon formation which is undesirable and steam is also necessary for the water gas shift reaction. Normally a steam to Carbon mole ration of 1:1 or 2:1 is used to prevent carbon formation.

It may be desirable to provide the syngas 2084 to a water gas shift 244 at step 124 to adjust the H₂ to CO ratio. In some examples, the H₂ to CO ratio may be adjusted to 3 (or a target of 3). When the H₂ to CO ratio is approximately 3, the syngas 208 can be readily converted to CH₄ in downstream methanators 246 (3H₂ reacts with one CO to form one CH₄ and H₂O vapor). Optionally, step 124 may include providing the syngas 208 to a first water gas shift 244 and then to a second water gas shift 244. In this example, the first water gas shift 244 may operate at a temperature between

310° C. and 450° C. and the second water gas shift 244 may operate at a temperature between 200° C. and 250° C. Higher CO conversion to H₂ occurs thermodynamically the lower the temperature.

It may be desirable to provide the syngas to a methanator 246 at step 126 to turn the syngas 2085 into a methane rich syngas 210. In some examples, the methanator may use a nickel (Ni) catalyst in 1-3 stages. Optionally, methanation may occur at pressure between 16-28 barg.

In some examples, when the syngas 208 is discharged by the pyrolysis kiln 204 and is refined as outlined above, the resulting methane rich gas 210 may have a CH₄ composition of 30% to 75% by volume In some examples, the remaining CO₂ within the methane rich gas 210 may be removed via amine or physical solvent scrubbing solutions, pressure swing adsorption, membrane processes, and/or other upgrading techniques to produce pipeline quality renewable natural gas.

### General Description of a Pyrolysis Kiln

As described above, at step 108 of method 100, biomass 202 may be provided to a pyrolysis kiln 204 in which it can be converted into at least one of biocarbon 206, syngas 208, and bio-oil.

Referring to FIG. 3, a specific example of a pyrolysis kiln 204 is shown. It is to be understood that method 100 may be performed using a different pyrolysis kiln 204 from that shown in FIG. 3.

In the example shown in FIG. 3, the pyrolysis kiln 204 extends longitudinally from an upstream kiln end 252 to a downstream kiln end 254. In the example shown in FIG. 3, the outer surface of the pyrolysis kiln is an insulative layer 256.

As shown in FIGS. 3 and 4, within the insulative layer 256, the pyrolysis kiln 204 includes a heat tube 258. As shown in FIG. 4, the heat tube 258 extends from an upstream heat tube 260 end to a downstream heat tube end 262 along a heat tube rotation axis 264.

Still referring to FIG. 3, the heat tube 258 has a heat tube inlet 266 at the upstream heat tube end 260 for receiving biomass 202 and a heat tube outlet 268 at the downstream end 262 for discharging at least one of biocarbon 206, syngas 208, and bio-oil. As shown in FIG. 4, the heat tube 258 has an inner heat tube surface 274 and an outer heat tube surface 278. In some examples, the heat tube 258 and the insulative layer 256 have the same length.

Still referring to FIG. 3, the pyrolysis kiln 204 may include a plurality of burners 276 which may be operable to heat the heat tube 258 and any biomass 202 contained therein. In some examples, the pyrolysis kiln 204 may have at least six burners 276. More specifically, in some examples, the pyrolysis kiln 204 may have at least three first burners 276₁ positioned on a first side 280 of the heat tube 258 and at least three second burners 276₂ positioned on a second side 282 of the heat tube 258. In the example illustrated in FIG. 3, the pyrolysis kiln 204 is shown to have seven first burners 276₁ positioned on the first side 280 of the heat tube. The pyrolysis kiln 204 illustrated also has seven second burners 276₂ positioned on the second side 282 of the heat tube which are not visible.

Referring now to FIG. 4, the first side 280 of the heat tube 258 and the second side 282 of the heat tube 258 are on opposite sides of a heat tube bisecting plane 286. The heat tube bisecting plane 286 is defined by gravity g and the heat tube rotation axis 264. That is, each of gravity g and the heat tube rotation axis 264 lie within the heat tube bisecting plane 268.

As shown in FIG. 4, in some examples, each of the first burners 276₁ and the second burners 276₂ may be positioned and oriented to apply heat to the heat tube 258 below the heat tube rotation axis 264. It may be desirable to apply heat below the heat tube rotation axis 264 because the biomass 202 within the heat tube 258 may be urged toward a lower end of the heat tube 258 due to gravity g as it passes through the heat tube 258. Positioning and orienting the burners 276 to apply heat to the heat tube 276 proximate the biomass 202 may promote more efficient heat transfer from the heat tube 258 to the biomass 202.

As described above, it may be desirable to rapidly increase the temperature of the biomass 202 when it enters the pyrolysis kiln 204. Accordingly, as shown in FIG. 3, in some examples at least one of the first burners 276₁ and at least one of the second burners 276₂ may be positioned proximate the heat tube inlet 266. In some examples, at least one of the first burners 276₁ and at least one of the second burners 276₂ may be positioned within 10% of a processing path length 288 away from the heat tube inlet 266. As used herein and in the claims, the processing path length 288 is the longitudinal distance between the heat tube inlet 266 and the heat tube outlet 268.

To promote heat application along the entire longitudinal length of the heat tube 258, the burners 276 may be longitudinally spaced apart from each other. For example, one of the first burners 276₁ may be longitudinally spaced more than 10% of the processing path length 288 away from an adjacent one of the first burners 276₁ and one of the second burners 276₂ may be longitudinally spaced more than 10% of the processing path length 288 away from an adjacent one of the second burners 276₂. In some embodiments, all of the first burners 276₁ are longitudinally spaced more than 10% of the processing path length 288 away from all of the other first burners 276₁ and all of the second burners 276₂ are longitudinally spaced more than 10% of the processing path length 288 away from all of the other second burners 276₂.

As shown in FIG. 3, in some examples, at least some of the burners 276 may be positioned in pairs along the pyrolysis kiln 204. Two burners, e.g., burners 276₁ₐ and 276_{1b}, may be considered a pair of burners 276 when they are located on the same side of pyrolysis kiln 204 and longitudinally spaced less than 5% of the processing path length 288 away from each other and each of those two burners 276₁ₐ, 276_{1b} are longitudinally spaced more than 10% of the processing path length 288 away from any other burner 276 on the same side of pyrolysis kiln 204.

As described above, it may be desirable to (a) heat the upstream portion 252 of the pyrolysis kiln 204 to an upstream kiln temperature between 800° C. and 1250° C. and (b) heat the downstream portion 254 of the pyrolysis kiln 204 to a downstream kiln temperature between 625° C. and 1000° C. In some embodiments, the downstream temperature is less than the upstream temperature (e.g. at least 50° C. less than the upstream temperature, such as 50° C. to 500° C. less than the upstream temperature). To do so, in some examples, each of the burners 276 of the pyrolysis kiln 204 may be individually thermal controlled for temperature or heat output rate (e.g., BTU's or MJ's). For example, each burner may have their own combustion fan that allows adjustment of the air to fuel ratio to that burner. In some examples, each pair of burners 276 may be thermally controlled with respect to adjacent burners 276 and/or pairs of burners 276. In alternative embodiments, burners 276 are not individually thermally controlled. For example, all of burners 276 may operate at substantially the same temperature or heat output rate.

In some embodiments, a subset of burners 276 on the same side of kiln 204 may form a heat zone. The burners 276 belonging to the heat zone may be thermally controlled independent of other burners 276, and may be thermally controlled as a group (e.g. to the same temperature and/or heat output rate). For example, each side of kiln 204 may have burners 276 grouped into two or more heat zones (e.g. 2-4 heat zones). In other embodiments, pyrolysis kiln 204 has only one heat zone.

Heat tube 258 may be made of any material suitable for withstanding the operating temperature and other conditions. Such conditions may include, for example a reducing atmosphere inside the heat tube 258 and an oxidizing atmosphere on the outside of the heat tube 258. In some examples, the heat tube 258 may be made of a high nickel alloy (e.g. high nickel and chromium alloy). As used herein and in the claims, a high nickel alloy is an alloy with typically 31 to 72 wt % nickel and high chromium of 17 to 27 wt %. Optionally, the high nickel alloy may include one or many other minor elements that provide oxidation and carburization resistance. For example, the heat tube 258 may be made of 330, 333, Inconel®-600, Inconel®-601, 602CA, or higher nickel alloys such as Hastelloy® X, 718, or INCOLOY® 800 H/HT. It may be desirable to make the heat tube 258 out of a high nickel alloy as such a material may be oxidation and carburization resistant. In other embodiments, heat tube 258 is not made of a high nickel alloy.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative of the invention and non-limiting and it will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

### Items

Item1: A method of processing biomass, the method comprising:
providing biomass to a pyrolysis kiln, the biomass having a moisture content of less than 40% and ash content of less than 50%, the biomass having a biomass input temperature of less than 125° C.;
heating at least an upstream portion of the pyrolysis kiln to maintain an upstream kiln temperature of between 800° C. and 1250° C., and feeding the biomass through the pyrolysis kiln for a pyrolysis duration of 5 to 60 minutes to produce an kiln output comprising biocarbon and syngas, the biomass increasing from the biomass input temperature to a biomass reaction temperature of at least 800° C. in less than 25% of the pyrolysis duration; and
discharging at least the biocarbon and the syngas from the pyrolysis kiln.

Item2: The method of any preceding item, wherein the kiln output, has an output composition that comprises:
greater than 19% by weight biocarbon,
greater than 50% by weight syngas, and
less than 20% by weight bio-oil.

Item3: The method of any preceding item, wherein the upstream kiln temperature is between 900° C. and 1,000°C.

Item 4: The method of any preceding item, wherein the pyrolysis duration is 5 to 30 minutes.

Item5: The method of any preceding item, further comprising
providing the biomass to a dryer upstream of the pyrolysis kiln; and
drying the biomass to the moisture content of less than 15%.

Item6: The method of any preceding item, further comprising
providing the biomass to a hammermill upstream of the pyrolysis kiln; and
fragmenting the biomass into biomass particles having an average biomass particle size between 1 mm and 25 mm.

Item7: The method of any preceding item, wherein the average biomass particle size is between 1 mm and 13 mm.

Item8: The method of any preceding item, wherein the average biomass particle size is between 1 mm and 3 mm.

Item9: The method of any preceding item, further comprising
providing the biomass to a pelletizer downstream of the mill and upstream of the pyrolysis kiln; and
pelletizing the biomass into biomass pellets having an average biomass pellet size between 5 mm and 7 mm.

Item10: The method of any preceding item, wherein heating at least an upstream portion of the pyrolysis kiln further comprises heating a downstream portion of the pyrolysis kiln containing the biomass to maintain a downstream kiln temperature of between 625° C. and 1000° C. and wherein the downstream kiln temperature is less than the upstream kiln temperature.

Item11: The method of any preceding item, wherein the downstream kiln temperature is at least 50° C. less than the upstream kiln temperature.

Item12: The method of any preceding item, wherein the downstream kiln temperature is between 625° C. and 850° C.

Item13: The method of any preceding item, further comprising feeding the syngas to an olivine reactor as reactor input syngas.

Item14: The method of any preceding item, wherein the olivine reactor is heated to maintain a reactor temperature of between 850° C. and 1250° C. to produce reactor output syngas, the reactor output syngas having an output H₂ concentration that is at least 200% of an input H₂ concentration of the reactor input syngas.

Item15: The method of any preceding item, wherein the olivine reactor contains calcined and H₂ reduced olivine.

Item16: The method of any preceding item, wherein the pyrolysis kiln contains olivine.

Item17: A method of processing biomass, the method comprising:
providing biomass to a pyrolysis kiln, the biomass having a moisture content of less than 40% and ash content of less than 50%;
heating at least an upstream portion of the pyrolysis kiln containing the biomass to maintain an upstream kiln temperature of between 800° C. and 1250° C., and feeding the biomass through the pyrolysis kiln for a pyrolysis duration of 5 to 60 minutes to produce a kiln output comprising at least biocarbon and syngas,
   the kiln output, excluding any ash, having an output composition comprising greater than 19% by weight biocarbon, greater than 50% by weight syngas, and less than 20% by weight bio-oil,
discharging at least the biocarbon and the syngas from the pyrolysis kiln.

Item18: The method of any preceding item, wherein the upstream kiln temperature is between 900° C. and 1,000°C.

Item 19: The method of any preceding item, wherein the pyrolysis duration is 5 to 30 minutes.

Item20: The method of any preceding item, further comprising
providing the biomass to a dryer upstream of the pyrolysis kiln; and
drying the biomass to a moisture content of less than 15%.

Item21: The method of any preceding item, further comprising
providing the biomass to a hammermill upstream of the pyrolysis kiln; and
fragmenting the biomass into biomass particles having an average biomass particle size between 1 mm and 25 mm.

Item22: The method of any preceding item, wherein the average biomass particle size is between 1 mm and 13 mm.

Item23: The method of any preceding item, wherein the average biomass particle size is between 1 mm and 3 mm.

Item24: The method of any preceding item, further comprising
providing the biomass to a pelletizer downstream of the mill and upstream of the pyrolysis kiln; and
pelletizing the biomass into biomass pellets having an average biomass pellet size between 5 mm and 7 mm.

Item25: The method of any preceding item, wherein heating at least an upstream portion of the pyrolysis kiln further comprises heating a downstream portion of the pyrolysis kiln containing the biomass to maintain a downstream kiln temperature of between 625° C. and 1000° C. and wherein the downstream kiln temperature is less than the upstream kiln temperature.

Item26: The method of any preceding item, wherein the downstream kiln temperature is at least 50° C. less than the upstream kiln temperature.

Item27: The method of any preceding item, wherein the downstream kiln temperature is between 625° C. and 850° C.

Item28: The method of any preceding item, further comprising feeding the syngas to an olivine reactor as reactor input syngas.

Item29: The method of any preceding item, wherein the olivine reactor is heated to maintain a reactor temperature of between 850° C. and 1250° C. to produce reactor output syngas, the reactor output syngas having an output H₂ concentration that is at least 200% of an input H₂ concentration of the reactor input syngas.

Item30: The method of any preceding item, wherein the olivine reactor contains calcined and H₂ reduced olivine.

Item31: The method of any preceding item, wherein the pyrolysis kiln contains olivine.

Item32: A method of processing biomass, the method comprising:
providing biomass to a pyrolysis kiln, the biomass having a moisture content of less than 40% by weight and an ash content less than 50%;
heating at least an upstream portion of the pyrolysis kiln containing the biomass to maintain an upstream kiln temperature of between 800° C. and 1250° C., and feeding the biomass through the pyrolysis kiln for a pyrolysis duration of 5 to 60 minutes to produce a kiln output comprising biocarbon and syngas; and
feeding the syngas to an olivine reactor as reactor input syngas, the reactor input syngas having an input H₂ concentration; and
heating the olivine reactor containing the syngas to maintain a reactor temperature of between 850° C. and 1250° C. to produce reactor output syngas, the reactor output syngas having an output H₂ concentration that is at least 200% of the input H₂ concentration.

Item33: The method of any preceding item, further comprising calcining olivine contained within the olivine reactor before feeding the syngas to the olivine reactor as the reactor input syngas.

Item34: The method of any preceding item wherein the pyrolysis kiln comprises olivine.

Item35: A pyrolysis kiln comprising:
a heat tube having an outer heat tube surface, the heat tube extending longitudinally from an upstream heat tube end to a downstream heat tube end along a heat tube rotation axis, the heat tube having a heat tube inlet at the upstream heat tube end and a heat tube outlet at the downstream heat tube end, the heat tube rotation axis and gravity direction defining a heat tube bisecting plane;
at least three first burners longitudinally spaced apart on a first side of the heat tube bisecting plane, the first burners positioned and oriented to apply heat to the heat tube below the heat tube rotation axis; and
at least three second burners longitudinally spaced apart on a second side of the heat tube bisecting plane, the second burners positioned and oriented to apply heat to the heat tube below the heat tube rotation axis.

Item36: The pyrolysis kiln of any preceding item, wherein
the heat tube inlet is spaced a processing path length away from the heat tube outlet; and
one of the first burners and one of the second burners is longitudinally spaced less than 10% of the processing path length away from the heat tube inlet.

Item37: The pyrolysis kiln of any preceding item, wherein
the heat tube inlet is spaced a processing path length away from the heat tube outlet;
one of the first burners and one of the second burners is longitudinally spaced less than 5% of the processing path length away from the heat tube inlet; and
one of the first burners is longitudinally spaced more than 15% of the processing path length away from an adjacent one of the first burners and one of the second burners is longitudinally spaced more than 15% of the processing path length away from an adjacent one of the second burners.

Item38: The pyrolysis kiln of any preceding item, wherein
each burner of the at least three first burners is arranged sequentially in a row; and
each burner of the at least three second burners is arranged sequentially in a row.

Item39: The pyrolysis kiln of any preceding item, wherein
the at least three first burners comprises at least seven first burners; and
the at least three second burners comprises at least seven second burners.

Item40: The pyrolysis kiln of any preceding item, wherein
the at least seven first burners comprises three pairs of first burners, the first burners of each pair of first burners are longitudinally spaced less than 5% of the processing path length away from the first burners of that pair of first burners and adjacent pairs of first burners are longitudinally spaced more than 10% of the processing path length away from each other; and
the at least seven second burners comprises three pairs of second burners,
the second burners of each pair of second burners are longitudinally spaced less than 5% of the processing path length away from the second burners of that pair of second burners and adjacent pairs of second burners are longitudinally spaced more than 10% of the processing path length away from each other.

Item41: The pyrolysis kiln of any preceding item, wherein
at least one of the first burners is individually thermal controlled relative to at least another one of the first burners; and
at least one of the second burners is individually thermal controlled relative to at least another one of the second burners.

Item42: The pyrolysis kiln of any preceding item, wherein the heat tube is made of a high nickel and chromium alloy.

1. A method of processing biomass, the method comprising:
providing biomass to a pyrolysis kiln, the biomass having a moisture content of less than 40% and ash content of less than 50%, the biomass having a biomass input temperature of less than 125° C.;
heating at least an upstream portion of the pyrolysis kiln to maintain an upstream kiln temperature of between 800° C. and 1250° C., and feeding the biomass through the pyrolysis kiln for a pyrolysis duration of 5 to 60 minutes to produce an kiln output comprising biocarbon and syngas, the biomass increasing from the biomass input temperature to a biomass reaction temperature of at least 800° C. in less than 25% of the pyrolysis duration; and
discharging at least the biocarbon and the syngas from the pyrolysis kiln.

2. The method of claim 1, wherein the kiln output,, has an output composition that comprises:
greater than 19% by weight biocarbon,
greater than 50% by weight syngas, and
less than 20% by weight bio-oil.

3. The method of claim 1, wherein the upstream kiln temperature is between 900° C. and 1,000° C.

4. The method of claim 1, wherein the pyrolysis duration is 5 to 30 minutes.

5. The method of claim 1, further comprising
providing the biomass to a dryer upstream of the pyrolysis kiln; and
drying the biomass to the moisture content of less than 15%.

6. The method of claim 1, further comprising
providing the biomass to a hammermill upstream of the pyrolysis kiln; and
fragmenting the biomass into biomass particles having an average
biomass particle size between 1 mm and 25 mm.

7. The method of claim 6, wherein the average biomass particle size is between 1 mm and 13 mm.

8. The method of claim 6, further comprising
providing the biomass to a pelletizer downstream of the mill and upstream of the pyrolysis kiln; and
pelletizing the biomass into biomass pellets having an average biomass pellet size between 5 mm and 7 mm.

9. The method of claim 1, wherein heating at least an upstream portion of the pyrolysis kiln further comprises heating a downstream portion of the pyrolysis kiln containing the biomass to maintain a downstream kiln temperature of between 625° C. and 1000° C. and wherein the downstream kiln temperature is less than the upstream kiln temperature.

10. A method of processing biomass, the method comprising:
providing biomass to a pyrolysis kiln, the biomass having a moisture content of less than 40% and ash content of less than 50%;
heating at least an upstream portion of the pyrolysis kiln containing the biomass to maintain an upstream kiln temperature of between 800° C. and 1250° C., and feeding the biomass through the pyrolysis kiln for a pyrolysis duration of 5 to 60 minutes to produce a kiln output comprising at least biocarbon and syngas,
the kiln output, excluding any ash, having an output composition comprising greater than 19% by weight biocarbon, greater than 50% by weight syngas, and less than 20% by weight bio-oil,
discharging at least the biocarbon and the syngas from the pyrolysis kiln.

11. The method of claim 10, wherein the upstream kiln temperature is between 900° C. and 1,000° C.

12. The method of claim 10, wherein the pyrolysis duration is 5 to 30 minutes.

13. The method of claim 10, further comprising
providing the biomass to a dryer upstream of the pyrolysis kiln; and
drying the biomass to a moisture content of less than 15%.

14. The method of claim 10, further comprising
providing the biomass to a hammermill upstream of the pyrolysis kiln; and
fragmenting the biomass into biomass particles having an average biomass particle size between 1 mm and 25 mm.

15. The method of claim 10, wherein heating at least an upstream portion of the pyrolysis kiln further comprises heating a downstream portion of the pyrolysis kiln containing the biomass to maintain a downstream kiln temperature of between 625° C. and 1000° C. and wherein the downstream kiln temperature is less than the upstream kiln temperature.

16. The method of claim 15, wherein the downstream kiln temperature is at least 50° C. less than the upstream kiln temperature.

17. The method of claim 10, wherein the pyrolysis kiln contains olivine.

18. A method of processing biomass, the method comprising:
providing biomass to a pyrolysis kiln, the biomass having a moisture content of less than 40% by weight and an ash content less than 50%;
heating at least an upstream portion of the pyrolysis kiln containing the biomass to maintain an upstream kiln temperature of between 800° C. and 1250° C., and feeding the biomass through the pyrolysis kiln for a pyrolysis duration of 5 to 60 minutes to produce a kiln output comprising biocarbon and syngas; and
feeding the syngas to an olivine reactor as reactor input syngas, the reactor input syngas having an input H₂ concentration; and
heating the olivine reactor containing the syngas to maintain a reactor temperature of between 850° C. and 1250° C. to produce reactor output syngas, the reactor output syngas having an output H₂ concentration that is at least 200% of the input H₂ concentration.

19. The method of claim 18, further comprising calcining olivine contained within the olivine reactor before feeding the syngas to the olivine reactor as the reactor input syngas.

20. The method of claim 18 wherein the pyrolysis kiln comprises olivine.
